(19) United States
(12) Patent Application Publication
(10) Pub. No.: US 2025/0032300 A1
(43) Pub. Date: Jan. 30, 2025

(54) ORTHOTIC BARS AND SYSTEMS FOR CORRECTING CLUBFOOT

(71) Applicant: MD Orthopaedics, Inc., Wayland, IA (US)

(72) Inventors: Lucas J. Mitchell, Wayland, IA (US); Marcus William Powell, New London, IA (US); Shawn Edward Twyman, Mt. Pleasant, IA (US); Jeffrey Allan Dassow, Fairfield, IA (US)

(21) Appl. No.: 18/714,845

(22) PCT Filed: Nov. 30, 2022
(86) PCT No.: PCT/US2022/051444

§ 371 Date: May 30, 2024

Related U.S. Application Data
(60) Provisional application No. 63/340,204, filed on May 10, 2022.
(60) Provisional application No. 63/284,416, filed on Nov. 30, 2021.
(60) Provisional application No. 63/284,411, filed on Nov. 30, 2021.

(51) Int. Cl. A61F 5/14; A43B 7/14; A61F 5/01

(52) U.S. Cl. CPC A61F 5/14; A43B 7/14; A61F 5/0111

(57) ABSTRACT

An assembly for locating footwear to maintain a user's feet in a predetermined therapeutic position, including a bar, typically a unitary curved bar forming an uninterrupted arc, where the bar has a first end and a second end, a first inner locating position on the first end, a first outer locating position on the first end, a second inner locating position on the second end, and a second outer locating position on the second end. Each of the locating positions typically includes a plurality of spaced apart ribs at least partially surrounding in a radial arrangement extending about a center point of a connection hole.

### CROSS-REFERENCE TO RELATED APPLICATIONS

This application is an International Patent Application that claims priority to and incorporates by reference in their entireties each of the following: U.S. Provisional Application Ser. No. 63/284,411, filed Nov. 30, 2021, entitled “UNITARY INJECTION MOLDED BAR FOR CORRECTING CLUBFOOT;” U.S. Provisional Application Ser. No. 63/284,416, filed Nov. 30, 2021, entitled “REVERSIBLE AND SELF-CONTAINED TENSIONING DEVICE;” and U.S. Provisional Application No. 63/340,204, filed on May 10, 2022, entitled “UNITARY INJECTION MOLDED BAR FOR CORRECTING CLUBFOOT.”

### BACKGROUND

Dr. Ignacio Ponseti was an internationally famous physician and surgeon specializing in the treatment and management of a childhood deformity commonly known as a clubfoot. For many decades Dr. Ponseti promoted the use of a foot and ankle abduction device, or orthosis, to correct and prevent relapses of the clubfoot deformity. These abduction devices typically consist of a linear, rigid bar connected between shoes worn by the child which the bar separates the feet of the child and holds the feet in an outward rotation or abduction. Typically, after casting is done to provide correction of clubfoot, this device is worn full-time for a period of months. The angle of outward rotation is typically set by the physician after treatment to retain the feet in the corrected position over time until no further retention is needed.

The technique for treating clubfoot developed by Dr. Ponseti has become known throughout the world and has been highly successful in treating club feet.

### SUMMARY

One aspect of the present disclosure includes an assembly for locating footwear to maintain a user's feet in a predetermined therapeutic position, including a bar having a first end and a second end, a first inner locating position on the first end, a first outer locating position on the first end, a second inner locating position on the second end, and a second outer locating position on the second end. Each of the locating positions includes ribs at least partially surrounding in a radial arrangement extending about a center point of a connection hole.

Another aspect of the present disclosure includes an assembly for locating footwear to maintain a user's feet in a predetermined therapeutic position, including a bar having a first end and a second end, a first locating position on the first end, and a second locating position on the second end. Each of the first and second locating positions includes a plurality of ribs at least partially surrounding in a radial arrangement extending about a center point of a connection hole. The assembly further includes first and second quick release members each having a plurality of ribs on a bottom side of the first quick release member that extend in a radial fashion about a stud extending downwardly from a base of the first quick release members. The ribs on each of the first and second quick release member are configured to fit between and interfere with the ribs at each of the first and second locating positions, respectively.

Yet another aspect of the present disclosure includes a method of assembling a bar assembly for the treatment of clubfoot, including attaching a first quick release member to a first end of a bar by inserting a first stud disposed on the first quick release member through one of a first hole at a first locating position and a second hole in a second locating position at the first end of the bar, attaching a second quick release member to a second end of the bar by inserting a second stud disposed on the second quick release member through one of a third hole at a third locating position and a fourth hole in a fourth locating position at the second end of the bar, angularly locating the first and second quick release members by the steps of rotating the quick release members until they reach a desired angular location, and tightening the quick release members to the bar, bringing release member ribs into interference with a set of bar ribs.

Another aspect of the present disclosure is generally directed to an orthotic brace for the treatment of clubfoot that includes: a bar assembly consisting of a first bar portion having a distal end and a second bar portion having a distal end wherein the first bar portion and the second bar portion are substantially planar and longer than they are wide and where the first bar portion and the second bar portion overlap at least a portion of one another and an overlapping portion is defined by the portions of the first bar portion and the second bar portion that overlap; a bar lock mechanism engaged to the overlapping portion of the bar assembly to fix the bar assembly at a user selected length; a first bar portion aperture through the first bar portion proximate the distal end of the first bar portion; a second bar portion aperture through the second bar portion proximate the distal end of the second bar portion; a first quick release member and a second quick release member each typically having an angular base portion and a quick release tab extending away from the base portion, which is typically an angular base portion, and configured to engage a receiving channel on the bottom surface of an orthotic shoe; a bottom mounting portion having a plurality of apertures therein; and an elastomeric intermediate portion having an engagement aperture through and a plurality of downwardly extending elastomeric extensions wherein the plurality of downwardly extending elastomeric extensions extend over an exterior surface of the bottom mounting portion, at least one of the plurality of aperture in the bottom mounting portion, or both. The first quick release member, the bottom mounting portion and the elastomeric intermediate portion and one of the first bar portion aperture and the second bar portion aperture are typically engaged with one another using a fastener, typically a threaded fastener such as a screw or bolt, that passes through each of the first quick release member, the elastomeric intermediate member, at least one of the first bar portion aperture and the second bar portion aperture and into a threaded engagement cavity on a bar assembly facing side of the bottom mounting portion.

These and other aspects, objects, and features of the present disclosure will be understood and appreciated by those skilled in the art upon studying the following specification, claims, and appended drawings.

### BRIEF DESCRIPTION OF THE DRAWINGS

In the drawings:

FIG. 1 is an elevated side view of an article of footwear according to an aspect of the present disclosure.

FIG. 2 is an elevated side view of opposite side of the shoe of FIG. 1 with the straps elevated for display purposes in a disengaged/unbuckled position.

FIG. 3 is a perspective view of the in-foot cradle of FIG. 1 according to an aspect of the present disclosure.

FIG. 4 is an elevated front view of the foot cradle insert of FIG. 3 according to an aspect of the present disclosure.

FIG. 5 is a top view of the foot cradle insert of FIG. 3 according to an aspect of the present disclosure.

FIG. 6 is a perspective view of a non-slip sock having slip resistant projections on a variety of the surfaces that engage the interior facing surfaces of the foot cradle insert and interior surfaces of the shoe not covered by the insert and also showing how a foot wearing the sock fits into the cradle of a shoe according to an aspect of the present disclosure.

FIG. 7 is a perspective view of a non-slip sock according to an aspect of the present disclosure positioned within the cradle of a shoe according to an aspect of the present disclosure.

FIG. 8 is a bottom perspective view of the sole portion of the shoe of FIG. 1 according to an aspect of the present disclosure.

FIG. 9 is a top, rear perspective view of a bar assembly according to an aspect of the present disclosure.

FIG. 10 is top view of a bar assembly of according to an aspect of the present disclosure.

FIG. 11 is a top view of a bar assembly according to an aspect of the present disclosure with a quick release member according to an aspect of the present disclosure affixed to each end thereof.

FIG. 12 is a bottom perspective view of a bar assembly of according to an aspect of the present disclosure.

FIG. 13 is a bottom view of a bar assembly according to an aspect of the present disclosure.

FIG. 14A is a rear elevation view of a bar assembly of according to an aspect of the present disclosure.

FIG. 14B is a front elevation view of the bar assembly of according to an aspect of the present disclosure with a quick release member affixed to each end thereof and showing the angle A of the angular base portion of the quick release member, which can be an angle from 10 to 20 degrees, more typically from 10 to 15 degrees.

FIG. 15 is a front elevation view of a bar assembly according to an aspect of the present disclosure.

FIG. 16 is a right-side elevation view of the bar assembly shown in FIG. 15.

FIG. 17 is a left-side elevation view of the bar assembly shown in FIG. 15.

FIG. 18 is a close-up top view of the circled end of the bar assembly identified by XVIII in

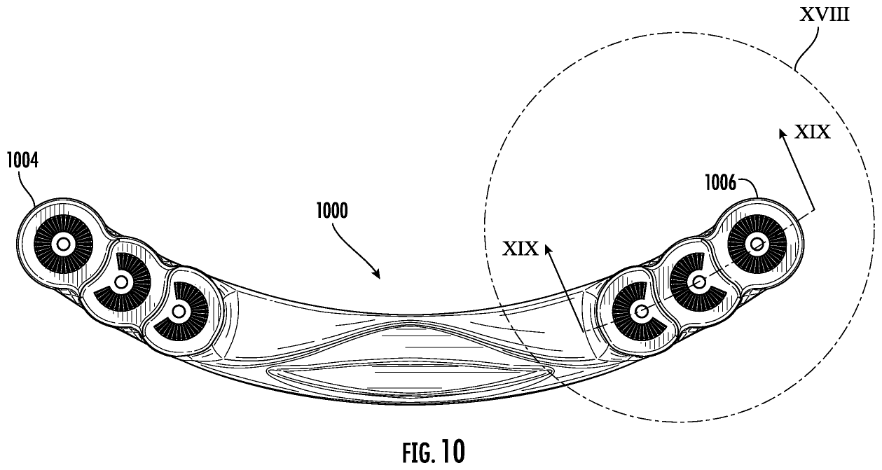

FIG. 10 according to an aspect of the present disclosure.

FIG. 19 is a cross-sectional view taken along line XIX-XIX of FIG. 10.

FIGS. 20A-20C are close-up views of an end of the bar assembly including a quick release member of an embodiment of the disclosure showing the quick release member in different possible and allowable positions.

FIG. 21 is a rear perspective view of a bar assembly according to an aspect of the present disclosure with a quick-release tabular attachment mechanism according to an aspect of the present disclosure attached to each end.

FIG. 22 is a front perspective view of a bar assembly according to an aspect of the present disclosure with a quick-release tabular attachment mechanism according to an aspect of the present disclosure attached to each end.

FIG. 23 is an enlarged perspective view of a first end of the bar assembly shown in FIGS. 21 and 22 showing angle demarcations for proper positioning of the shoes engaged with the bar.

FIG. 24 is an enlarged perspective view of a second end of the bar assembly shown in FIGS. 21 and 22 showing angle demarcations for proper positioning of the shoes engaged with the bar.

FIG. 25 is top view of the bar assembly shown in FIG. 21 according to an aspect of the present disclosure.

FIG. 26 is a right-side elevation view of the bar assembly shown in FIG. 21 according to an aspect of the present disclosure.

FIG. 27 is a front elevation view of the bar assembly shown in FIG. 21 according to an aspect of the present disclosure.

FIG. 28 is a bottom view of the bar assembly shown in FIG. 21 according to an aspect of the present disclosure.

FIG. 29 is a perspective view of a clip and bar system according to an aspect of the present disclosure that does not use a fastener to engage the clip to the bar.

FIG. 30 is a bottom perspective view of a clip and bar system according to an aspect of the present disclosure that does not use a fastener to engage the clip to the bar.

FIG. 31 is a top perspective view of a clip and bar system according to an aspect of the present disclosure where the quick clip attachment as a top quick clip engagement portion, an intermediate portion, which is typically a thermoplastic elastomeric material, and a bottom portion where the intermediate portion and the bottom portion engage the bar therebetween to retain the quick clip portion in position on the bar.

FIG. 32 is a bottom perspective view of a clip and bar system according to an aspect of the present disclosure where the quick clip attachment as a top quick clip engagement portion, an intermediate portion, which is typically a thermoplastic elastomeric material, and a bottom portion where the intermediate portion and the bottom portion engage the bar therebetween to retain the quick clip portion in position on the bar.

FIG. 33 is a perspective view of a first end of the bar assembly shown in FIG. 35 showing the attachable extensions to the bar.

FIG. 34 is a top plan view of a first end of the bar assembly shown in FIG. 35 showing the attachable extensions to the bar.

FIG. 35 is a bottom view of a first end of the bar assembly shown in FIG. 35 showing the attachable extensions to the bar.

FIG. 36 is an exploded, perspective view of a clip and bar system according to an aspect of the disclosure where the ends are extendable by attaching extensions to the ends of the bar.

### DETAILED DESCRIPTION

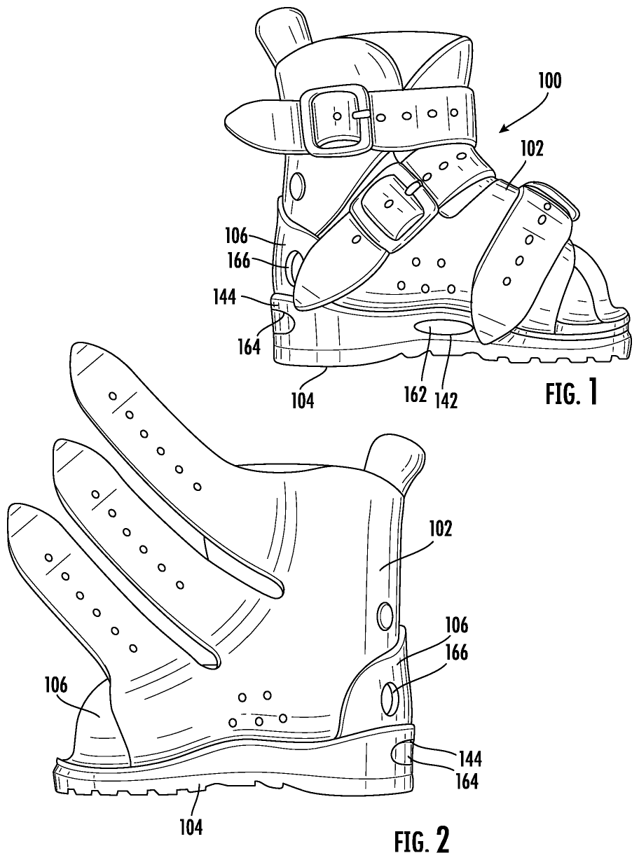

For purposes of description herein, the terms “upper,” “lower,” “right,” “left,” “rear,” “front,” “vertical,” “horizontal,” and derivatives thereof shall relate to the disclosure as oriented in FIG. 1. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

Now referring to the drawings wherein like numerals refer to like matter throughout, and more specifically referring to FIG. 1, there is shown an article of footwear 100, of the present disclosure. Footwear 100 is shown having an upper 102 which could be any type of wearable material, but is typically leather, fabric, vinyl or other material suitable for a shoe or sandal. Footwear 100 further includes a sole 104, which is typically a single molded piece of plastic material, such as a flexible polymer. Other material suitable for shoe or sandal soles could be used as well. Also shown in FIGS. 1-5 is insole 106, which is disposed above upper 102 and generally within upper 102. Typically, insole 106 is a single piece of molded polyurethane or rubber-like material. Typically, insole 106 is pliable enough so as to permit it to conform to the shape of a child's foot when inserted therein and secured thereon by upper 102. The insole 106 is also sufficiently soft and pliable to be comfortable for the child to wear without injury or irritating the child's skin.

As shown in FIGS. 1-5, insole 106 is typically secured to sole 104 by glue and may be supplemented, if necessary, providing an insole side protuberance 162 or a plurality of protuberances may extend through a sole side hole 142 in sole 104 or a corresponding number of side holes. Similarly, insole heel protuberance 164 or a plurality or protuberances may extend through sole heel hole 144 or holes and thereby hold insole 106 stable with respect to sole 104. The protuberances 162 and 164 are shown as having an elliptical cross-sectional shape, but could conceivably be any shape/design and they typically protrude away from the main surface of the insole 106. Insole 106 is also shown having an insole heel right viewing hole 166, which is located near the heel of the insole 106, so that it can readily be seen that a child's foot is in contact with the insole 106. In a preferred embodiment, the insole heel right viewing hole 166 is a pair of holes on opposite sides of the heel or is a single hole which spans to both sides of the heel. Any number of viewing holes can be used and a plurality of viewing holes on each side or a single hole on one side may be implemented. In either embodiment, the viewing hole(s) are typically positioned such that it would be possible to view the placement of the child's foot with respect to the insole 106 from a vantage point on either side of the heel.

Now referring to FIG. 2, there is shown an opposite side of footwear 100 that is shown FIG. 1 in an unbuckled position. The footwear 100 is shown using buckles, but the footwear 100 may be enclosed on a child's foot using another attachment system such as a hook and loop fastening system or even a tie system, but a tie system is not typically employed because they are harder than other attachment systems for the child of an age that typically uses the system to attach. Moreover, the hook and loop fastener engagement system is sometimes disadvantageous because young children may be able to open the footwear when they should not.

Now referring to FIG. 3, there is shown a perspective view of the insole 106 of FIG. 1 when it is separated from the upper 102 and sole 104 of the footwear 100. Insole 106 is shown having an insole toe end 302 and an insole sole foot side 304. Preferably, insole sole foot side 304 is generally a flat surface without substantial contours to fit to an arch of a foot. Insole 106 includes an insole heel and ankle support 306 with an insole strap support 308, which keeps an ankle support strap from slipping downward. Insole 106 further includes insole side wall 310 on opposing sides of insole sole foot side 304. In an aspect of the present disclosure, insole sole foot side 304, insole side wall 310 and insole heel and ankle support 306 all are portions of a single molded piece of urethane, pliable rubber-like material or flexible polymer material. The molded, but flexible polymer material is more comfortable for the younger children aged 0-5 years old that might typically wear the footwear during treatment of clubfoot.

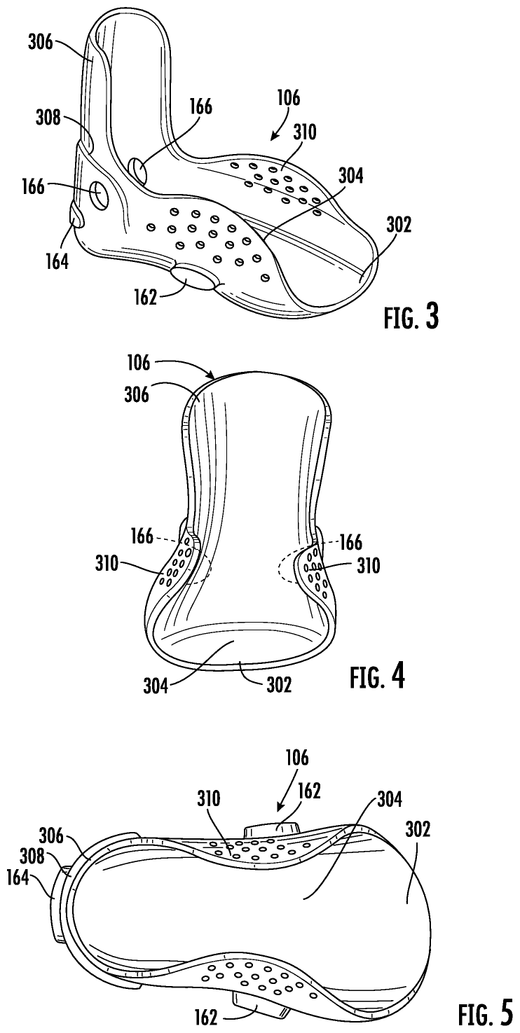

Now referring to FIGS. 4 and 5, there are shown front and top views, respectively of the insole 106 of FIGS. 1, 2 and 3 removed from the upper 102 and the sole 104.

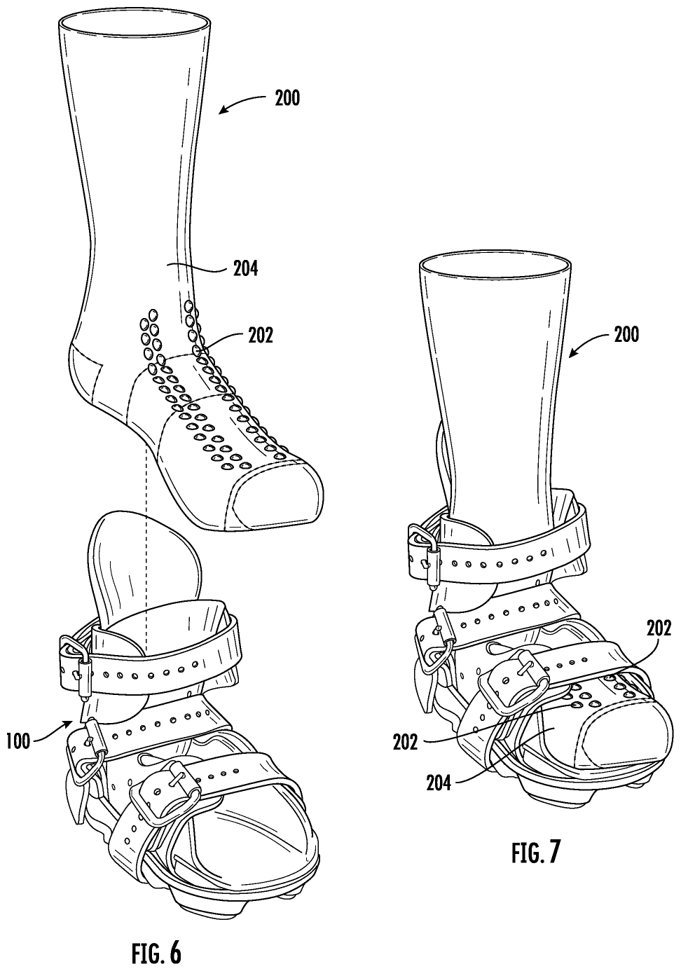

Now referring to FIGS. 6 and 7, a practitioner may determine that the user needs more restriction to the amount of foot movement within the footwear 100 or a user may wish to make sure the foot within the footwear 100 is less likely to move from a properly seated position within the footwear. As shown in FIG. 6, the user may use socks 200 that have a number of slip resistant nubs 202 attached to various surfaces thereof. The nubs 202 restrict the amount of movement of a user's foot within the footwear 100 using two methods. First, the nubs 202 are of a material that increases the coefficient of friction between the sock 200 and the shoe 100, specifically in the area of the nubs 202. Second, the nubs 202 extend outwardly from the base area 204 of the sock 200 in order to take up more room and create more interference between the sock 200 and the insole 106 of the shoe 100. The nubs 202 are typically placed such that the fewest number of nubs is needed to prevent relative movement between the sock 200 (and the user's foot) and the shoe 100. However, it should be known that any number of nubs may be used, and the nubs may be any shape. The shape is not critical. Typically, nubs are place on the bottom surface of the sock and the left and right sides of the top surface of the sock that covers the top of the foot when the socks are worn. Numbs can be placed in the exact center of the top and omitted from the bottom surface as an alternative example as well or place on the bottom surface and the top center surface too. They can be placed on the later side surfaces and optionally one or both of the top and bottom too. Often the nubs in some number or shape are positioned on the top of the sock and the bottom but can be place anywhere. However, typically the nubs engage a plurality of the interior facing surfaces of the orthotic shoe or shoe insert. The nubs, while shown as rounded dots, may be of a shape such as a diamond, a triangle rectangular strip etc. Typically, one selects a design that might be more attractive/“fun looking” to an infant or toddler in order to decrease the user's resistance to wearing the device.

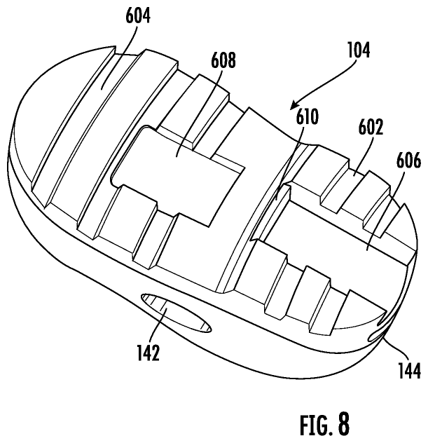

Now referring to FIG. 8, there is shown a perspective view of the bottom of sole 104 of the footwear 100 FIG. 1. Sole heel 602 is shown disposed at an opposite end of sole 104 from sole toe end 604. Sole heel 602 has a sole attachment receiving groove 606 therein, which is separated from quick release push button receiving void 608 by quick release button barrier 610. Preferably, all of these features, sole heel 602, sole toe end 604, sole attachment receiving groove 606, quick release push button receiving void 608 and quick release button barrier 610, are integrally formed in a single piece of durable plastic or other material suitable for a rigid shoe sole. The sole attachment receiving groove 606 and the quick release push button receiving void 608 may be each rectangularly shaped and aligned with one another; however, other shapes may be employed, but these portions are typically sized and shaped to engage the quick release clips discussed later therein. The material of the sole that establishes the sole attachment receiving groove 606 and the quick release push button receiving void 608 may need to be more rigid than a typical shoe sole material and substantially unbendable because of the need for increased lateral forces owing to the use of the splint between the shoes. The quick release push button engagement system of the present disclosure typically engages the quick release member, which has quick release button 708 that engages the quick release push button receiving void 608. This arrangement allows for the rapid and easy release of the shoes of the present disclosure from the clips by hand and without the use of tools-especially by an adult with stronger hand strength. The quick clip typically has a resistance to biasing that prevents a young child from disengaging the shoe from the clip of the present disclosure.

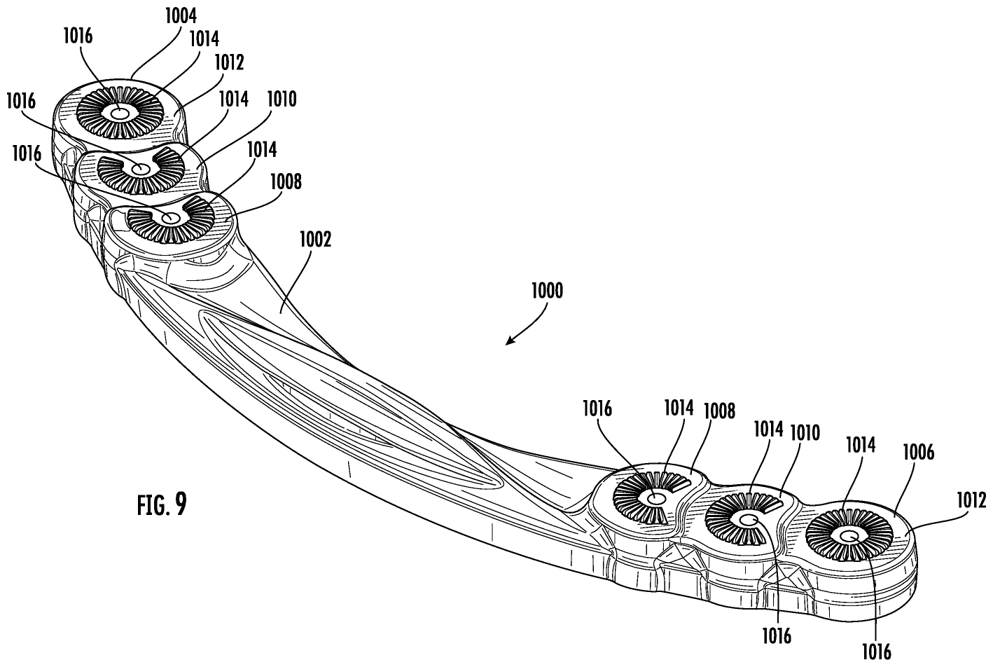

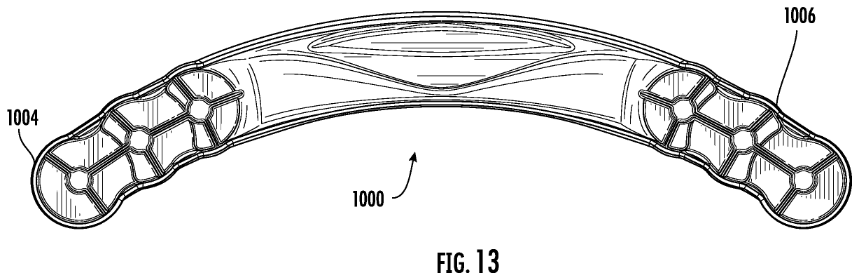

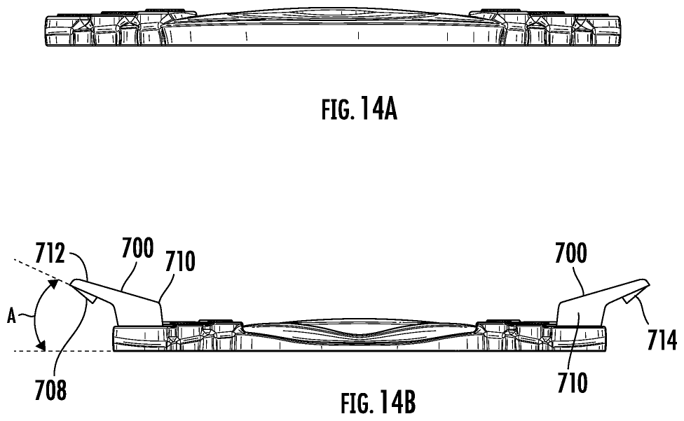

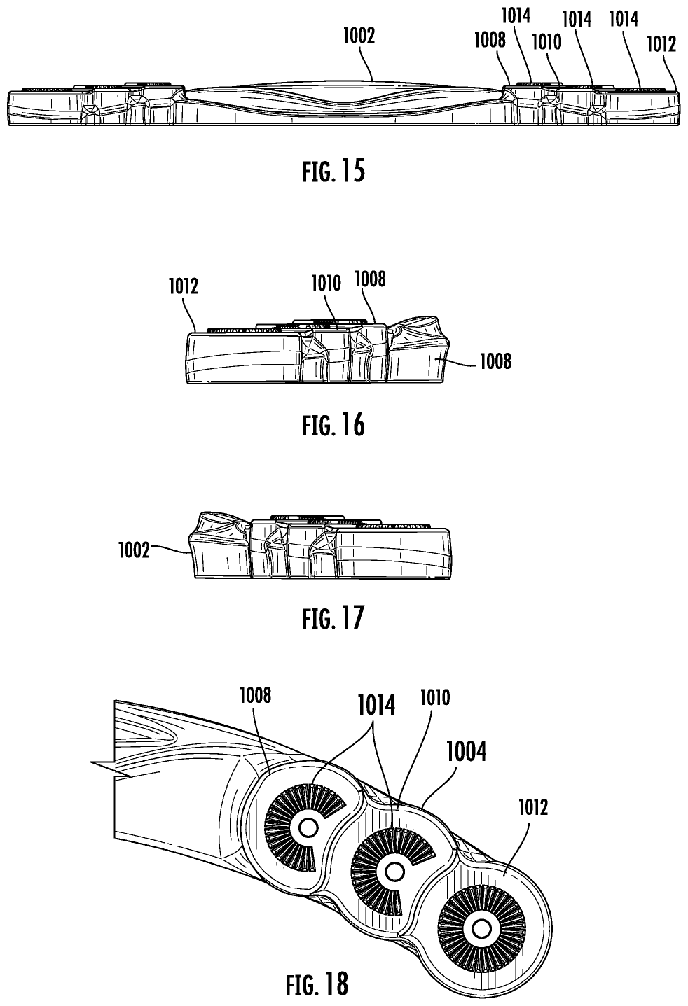

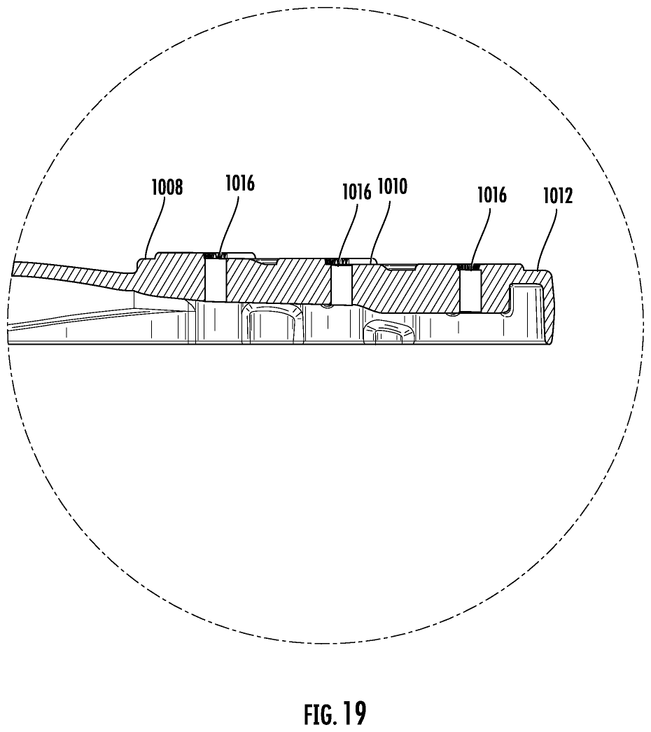

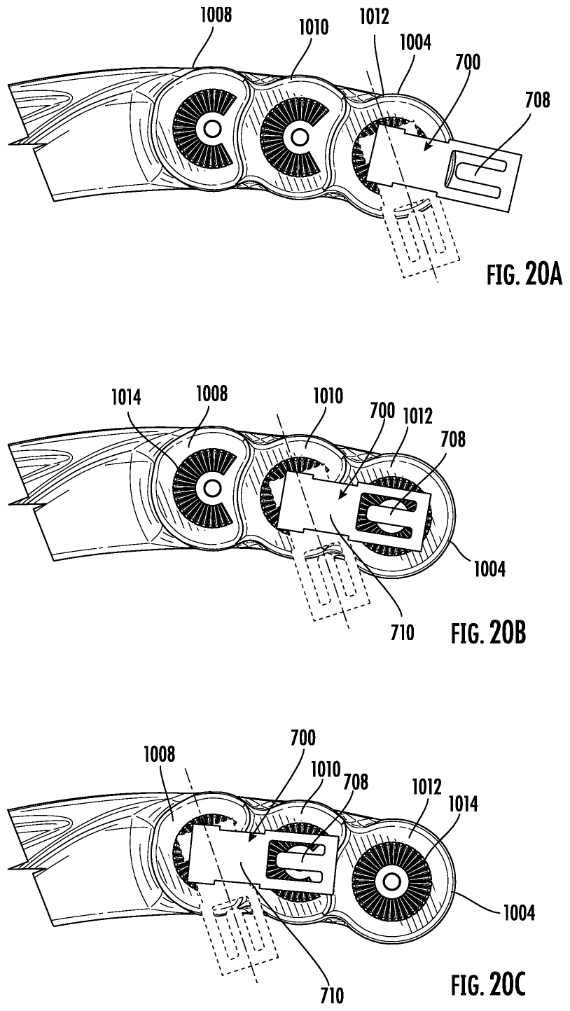

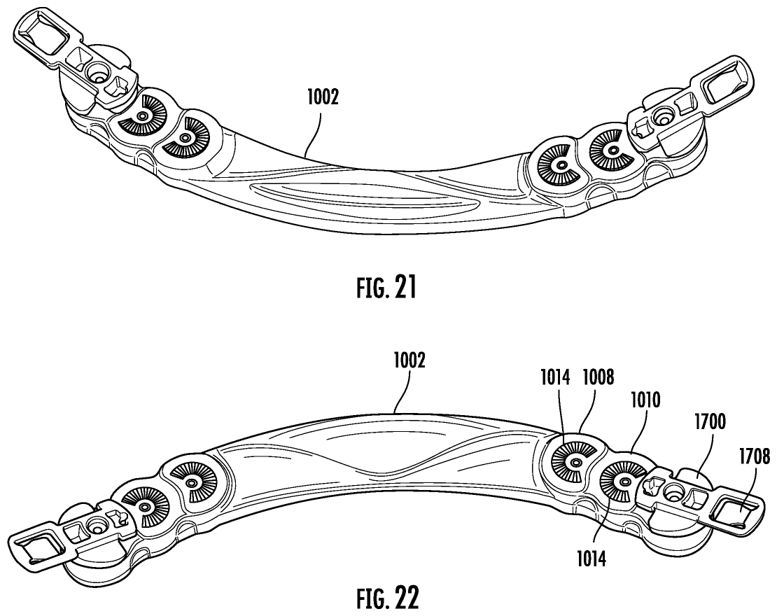

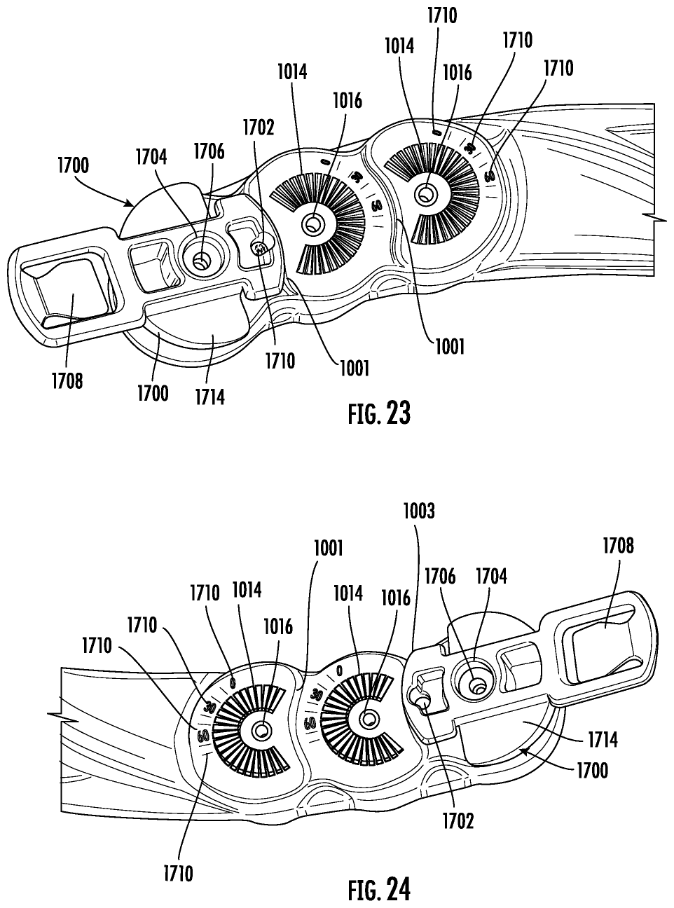

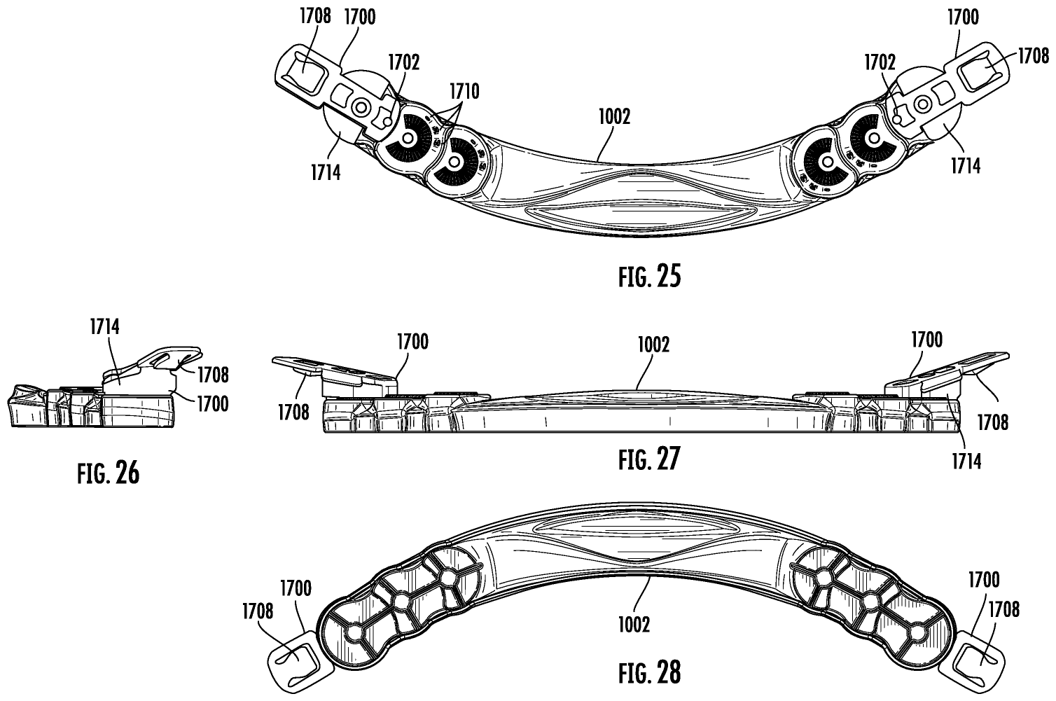

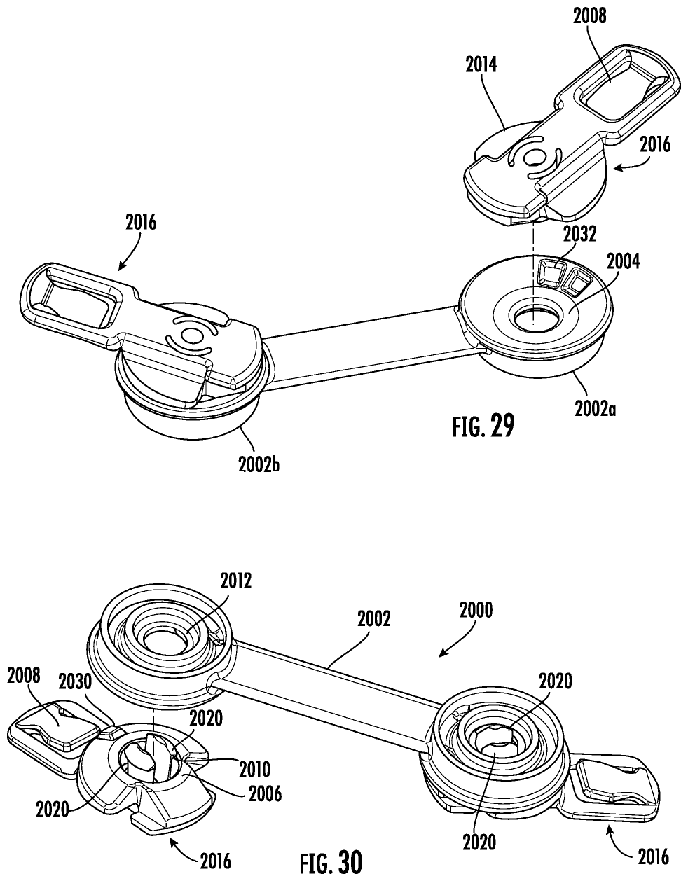

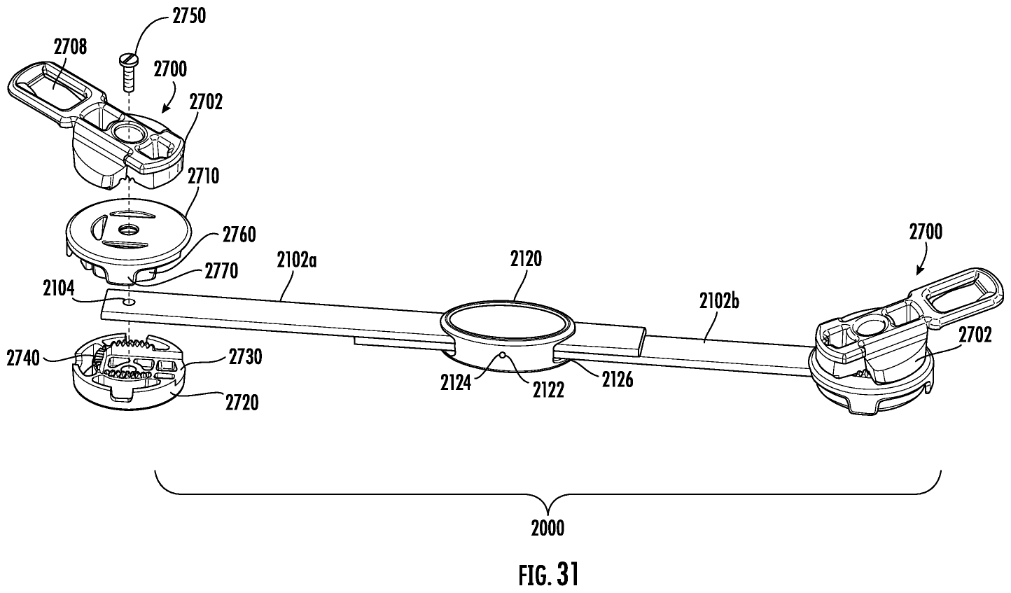

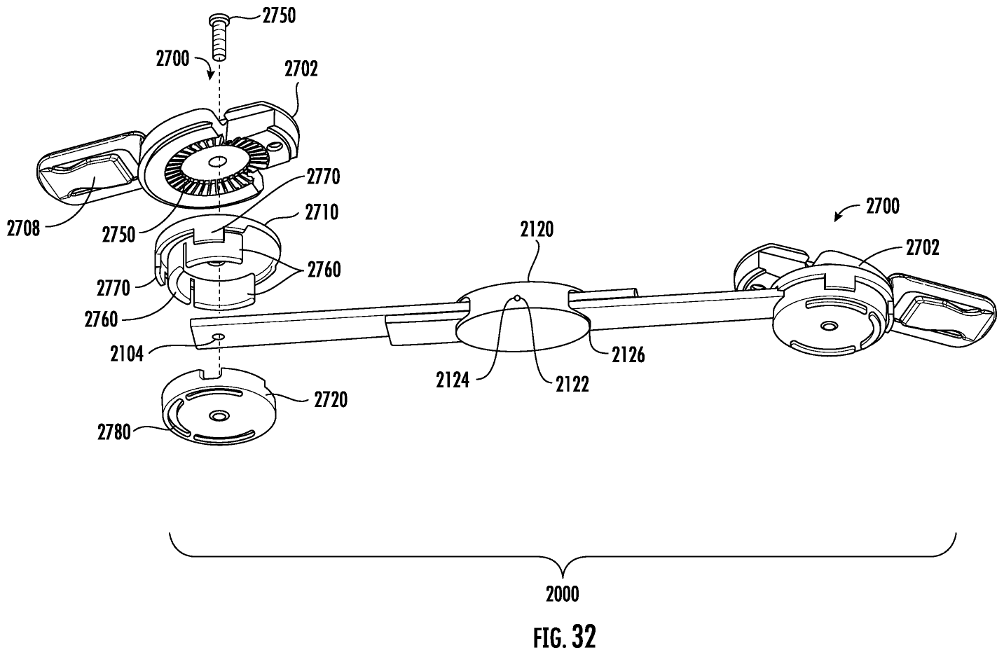

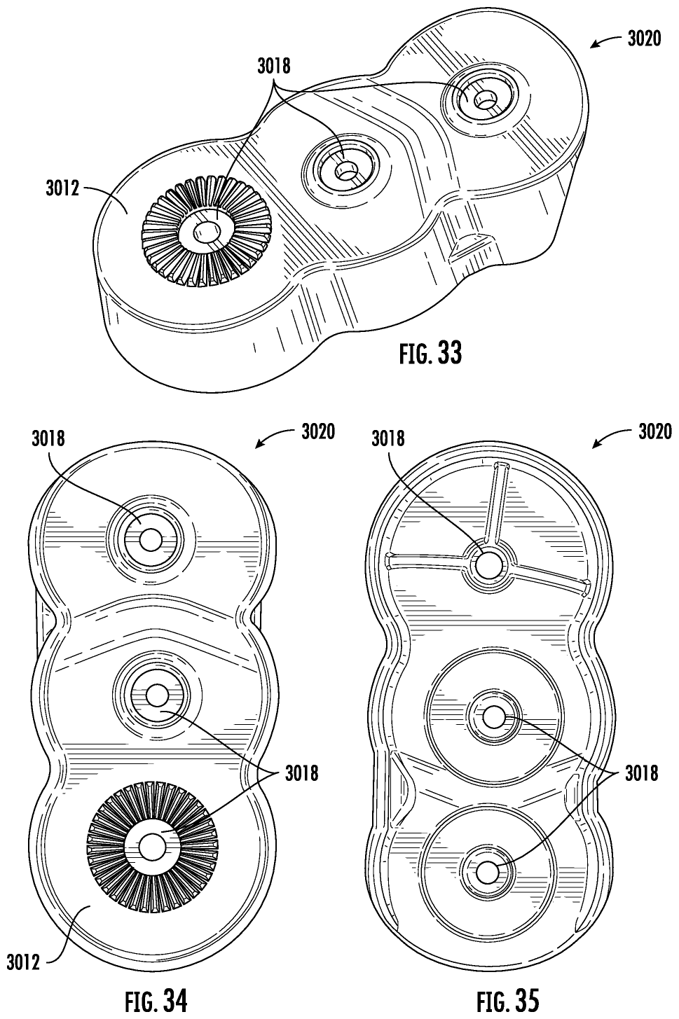

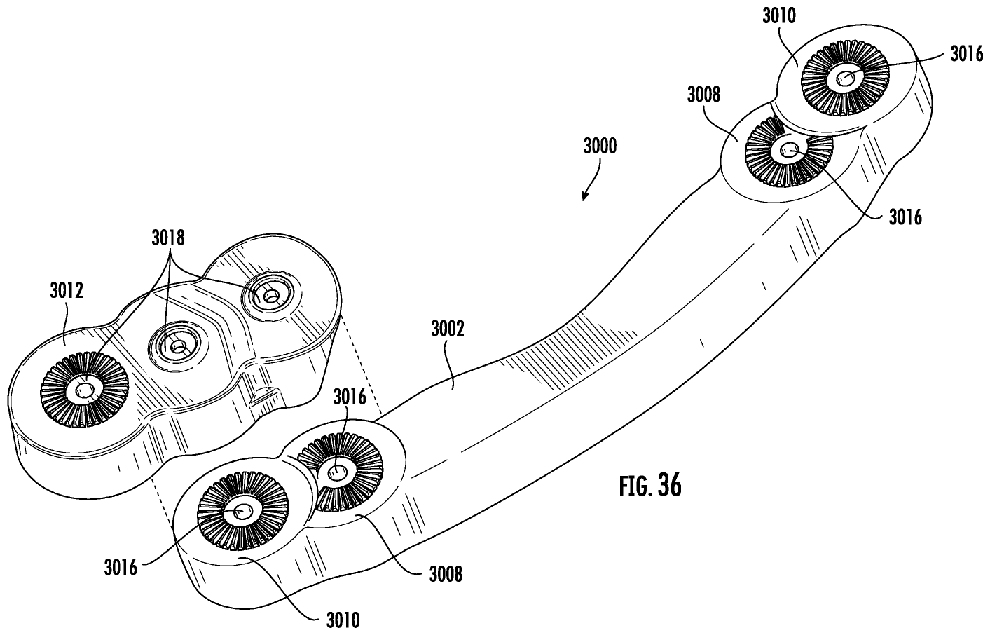

Now referring to FIGS. 9-20C, there is shown an adjustment bar assembly 1000. The bar assembly 1000 typically includes a bar 1002, and a pair of quick release members 700. The bar 1002 may include a first end 1004 and a second end 1006 and is typically a single unitary bar produced from a thermoplastic material, for example. Each of first and second ends 1004, 1006 may include a plurality of locating positions. FIG. 9 shows three locating positions, an inner locating position 1008, an intermediate locating position 1010, and an outer locating position 1012. The locating positions may be offset in height as shown in FIGS. 14A-15, or they may all share a plane, they all may be oblique to one another, or any other arrangement desired. Similarly, the second end may include an inner locating position 1008, an intermediate locating position 1010, and an outer locating position 1012. The inner location position spaces the pair of quick release members about 8 inches apart, the intermediate position about 10 inches apart and the outer locating position about 12 inches apart. This is sufficient for most patients needing clubfoot treatment. However, in rarer instances, further distance is needed. This may be accomplished by a fourth position being added to the unitary bar integral with the others. Alternatively, an extension bracket consisting of three circular portions substantially corresponding in shape and substantially the same archuous angle may be engaged to the outermost positions with the third position of the extension bracket extending beyond the end of the bar such that the quick release bracket engagement location is a fourth location about 14 inches apart from one another on each side of the bar. Typically, the center aperture of the third circular portion that forms the fourth engagement position is determined by a linear line from the inner locating position's center point through the middle circular portion of the bracket. When attached, two of bracket's circular portions are attached via screw engagement with the corresponding apertures of the outermost positions on the bar, the intermediate locating position 1010 and the out locating position 1012 with the third circular portion extending beyond the out locating position free for engagement with the quick release member.

On each of the locating positions, there may be ribs 1014 arrayed in a radial fashion about a connection hole 1016. It should be understood that the bar may include fewer or more locating positions 1012 to allow for more adjustability of the device for a user. Moreover, the greater number of ribs allows for more fine tuning of the position of the quick release member rotationally about the connection hole 1016. An alternative configuration that may be utilized to fine tune the angle of the locating position and thereby the shoe when engaged with the bar. In an alternative arrangement, at least two projections of any shape but having a thickness sufficient to have shear strength that prevents the projection from being sheared off when in an engaged position within an at least substantially correspondingly shaped cavity or correspondingly shaped cavity during use at a given angle. More typically, more than two spaced apart projections may be configured. In this embodiment or the prior embodiment, the projections could be on the bar and/or the bottom surface of the quick release member or bracket. Most typically, the spaced apart projections will be on the bar and the cavities on the bracket but this configuration may be inverted in whole or in part. According to this embodiment, a plurality of shear resistant projections is used and positioned either on the bar or the bottom surface of the bottom portion of a quick release member or other intermediate bar engagement bracket that may interface with a quick release member or other shoe engagement system. The shear resistant projections may be positioned in a spaced apart concentric configuration at the same radial distance from the center of the bar surface or bottom surface of the quick release member or may be spaced at different radial distances away to allow for increased flexibility in the number of positions that may be implemented. Typically, about 6 to 9 shear resistant projections are used. In operation, only one of the shear resistant projections will be securely seated in an engagement cavity that corresponds with one of the plurality of engagement positions. Once moved out of engagement, the projection would next move circumferentially about the center axis and fall into engagement within a follow-on cavity that carries the projection within it through all of the other engagement position/angles where one other projection is engaged at a time until the shear resistant projection again is positioned at the angle corresponding to its specific engagement cavity.

The quick release member is typically attached using solely the connection hole 1016. Each connection hole typically includes a threaded metal sleeve positioned within the aperture that typically engages a screw through the quick release member and into the threaded receiving aperture. The female threaded metal sleeve positioned within the aperture is typically formed by molding the bar 1000 around the metal threaded screw receiving aperture. In an aspect of the present disclosure, the bar 1000 is typically a single unitary bar molded from a thermoplastic material such as polyethylene, polypropylene or polyvinyl chloride, that allows the bar to be relatively light, while offering the strength necessary to keep the user's feet in a desired location needed for the treatment of clubfoot according to the Ponseti method. In this embodiment, the connection holes 1016 may include a threaded metal sleeve as mentioned above. The metal sleeve protects the thermoplastic material from abrasion that may occur as studs from the quick release members (detailed below) are inserted and removed from the connection holes 1016. The metal sleeves may be insert molded with the bar as a unitary structure. While the bar 1000 typically has connection holes 1016 to engage the quick release members via a screw engagement that passes through the quick release member 1700 (see FIG. 23, for example) or is engaged on a bottom surface of the quick release member, an alternative construction may be employed where the bar 1000 has a threaded projection that may be integrated into the bar instead of a connection hole. The threaded projection may be removable by hand and without the use of tools or with a screwdriver or other hand tool that allows the projection to be securely engaged with the bar at a selected location. The removability of such a projection may be done, to accommodate its use in multiple positions as contemplated in the bar described herein and shown in the drawings. Conceivably, a bar of the present disclosure may only have one attachment location with a projection at each end. This alternative construction is not typically employed but is possible. More typically, as discussed above and herein, a bar of the present disclosure will typically contain one or more aperture at each end with a screw or other attachment member extending into a selected aperture to retain the quick clip in position.

The adjustment bar assembly 1000 further typically includes quick release members 700, which are configured to be inserted into sole attachment receiving groove 606 and quick release push button receiving void 608 of FIG. 8. The quick release members 700 may have main body 712 that extends outwardly from a base portion 710. The quick release main body 712 is configured to fit into sole attachment receiving groove 606 and slide in further until quick release button 708 snaps into a locked position in quick release push button receiving void 608. Quick release button 708 is preferably biased outwardly or downwardly by a spring-like structure or arrangement, such as a living hinge or an external spring.

As shown in FIGS. 20A-C, in order to locate the quick release member 700, and by extension the footwear 100 of the user, the angular adjustment base 710 may include a plurality of ribs that are arrayed in a radial fashion about a bolt or stud that is coupled to the quick release member 700 on a bottom side of the base 710. The radial ribs extend outwardly in a direction that extends from the center of the bolt or stud such that the quick release member may be rotated about 360 degrees and the ribs still interface with the ribs 1014 on the bar itself as detailed below. The ribs 1014 are typically a plurality of radially extending upwardly extending members or elevated linear bumps that extend outwardly from the connection holes 1016.

The quick release members 700 may be attached to the adjustment bar 1000 at any one of the connection holes 1016. The quick release members 700 may include a threaded stud, a bolt, or other fastener that extends through the connection hole 1016, and is attached by a nut on the opposite side of the bar 1000. The nut is attached to the end of the stud and tightened until it applies pressure against the bottom side of the bar 1000, keeping the ribs 1014 of the bar and the correspondingly shaped ribs on the bottom of the quick release member 700 in contact and in interference with one another thereby preventing the quick release member from rotating when tightened fully. It should be noted that the bolt and nut combination may be reversed, i.e. the bolt may be integral with the bar 1000, or the bolt may be a completely separate member from the quick release member 700 and extend through a corresponding hole in the quick release member 700.

The ribs on the bottom side of the quick release member 700 correspond to the ribs 1014 on the top side of the bar 1000 at the locating positions. The ribs of the quick release member 700 fit between the ribs 1014 on the bar 1000. This fit between the ribs on the quick release member 700 and the ribs on the bar 1000 allow the quick release member 700 to be attached to the bar 1000 in any angular position around 360 degrees, and typically in very small increments enabling fine-tuned adjustments during treatments. The bottom surface of the top portion of the quick release member 700 typically has ridges that matingly engage the groves between the ribs 1014. Ridges on the bottom surface of a quick release member of the present disclosure is also shown in at least FIG. 32.

FIGS. 20A-20C show the adjustability that is available to a practitioner when customizing a fit to a given user. The angular adjustability of the quick release member 700, in combination with the various locating positions 1008, 1010, 1012, allows a practitioner to easily adjust both the spacing and the relative angles of the footwear 100 when worn by a user.

In operation, the present invention can be used to treat clubfoot in children, more typically used to carry out the technique of Dr. Ponseti to treat clubfoot using a process as described below.

A practitioner determines the proper spacing and angle for a patient's/user's feet using any method known in the art. The practitioner then attaches one of the quick release members 700 to the bar 1000 at one of the connection holes 1016 by inserting the integrally coupled bolt into one of the connection hole 1016. A nut is attached on the opposite side of the bar from the quick release member 700, but not tightened. The practitioner then orients the quick release member at the desired angle. The nut is then tightened down. As the bolt is tightened down, the ribs of the quick release member interfere with the ribs on the bar 1000, preventing rotation of the quick release member 700 with respect to the bar 1000. The practitioner then attaches the second quick release member 700 to the other end of the bar 1000 in the same way.

The practitioner then places the shoes or footwear 100 on the user's feet. Each of the shoes 100 are placed into engagement with the quick release members 700 by sliding the quick release main body into sole attachment receiving groove 606 on the footwear 100 and sliding in further until quick release button 708 snaps into a locked position in quick release push button receiving void 608. Once snapped into the locked position, as discussed above, the sole of the footwear is engaged to the quick release member 700 until the quick release member is biased by a person to a disengagement position and pulling the footwear out of engagement.

It should be noted that the attachment described above may further be accomplished in alternate steps without deviating from the spirit of the disclosure. For instance, the footwear 100 may be attached to the user first and quick release members 700 slid into their respective receiving grooves 606 before attachment to the bar. The practitioner may then insert the bolts of the quick release into the sole of the footwear and tighten down. Further, there may be no bolts at all, but rather a quick-click style attachment or something similar.

FIGS. 21-28 demonstrate another embodiment of the bar assembly 1000. As was the case with the previous aspects of the present disclosure discussed above, the bar 1002 may include at least three locating positions on either end, although there may be more or fewer locations on a single bar. The locating positions are typically positioned at different heights with all but the most exterior attachment location having a curved indented portion 1001 to accommodate the end 1003 of the quick release member opposite the snap engagement end of the quick release member that has the outwardly extending snaps 1708. A pair of quick release members 1700 may be attached to the bar 1002-one at each end of the bar 1002 at any of the locating positions, but typically the locating positions that are each equidistant form the center of the bar. As shown in FIG. 23-28, the quick release members 1700 may include snaps 1708 that allow the quick release members 1700 to attach and detach from the sole of a shoe quickly and easily.

Looking closer at FIGS. 23-28, the quick release members 1700 may further include wings 1714 that allow for more interference members to be included on the bottom side of the quick release members 1700. The interference members are typically radially extending teeth that protrude from the bottom surface of the quick release members including the wings 1714 about the engagement member, typically a screw or bolt, receiving aperture 1706 that extends through the quick release member 1700 to allow for the user to see the connection holes 1016. The top portion of the quick release member 1700 typically has a screw or bolt head receiving recess 1706 centered around the aperture 1704 such that the head of the screw, bolt or other typically threaded engagement member is received therein when fully engaged and not extending above the top surface plane of the quick release member 1700. This prevents the engagement member from catching on clothing or other items while in use. With more interference members interacting with each other, the practitioner may be more confident that the bar 1000 will keep the user's feet in the correct orientation when engaged with the quick release members that themselves are engaged with the bar 1000. The quick release member 1700 may also include a window 1702, and the bar 1002 may include angle markings 1710. The window may be a clear portion or, more typically, an aperture through the quick release member allowing the user to see through from the top of the quick release member to a surface underneath it. The angle marking 1710 as shown include marking for 0, 30, and 60 degrees, but they can be set for any increment and in any orientation that a practitioner sees fit. The particular angle markings shown for exemplary purposes only and may not be shown in the orientation that a practitioner wishes for any given user. The window 1702 may be of any shape, but is typically circular and allows the medical professional or clubfoot practitioner or parent to see the angle that the footwear and therefore the foot will be in when engaged with the quick release member and bar.

FIGS. 25-28 show complete orthographic projection views of the quick release members 1700 as attached to the unitary bar 1002. The quick release members 1700 may be set at an angle to a horizontal plane as generally defined by the bottom portion of the bar 1002 shown in FIG. 27. This angle sets the users pronation or supination angle while using the device. This angle may be defined by the quick release members themselves and may be set at any angle, but preferably about 10 degrees or 15 degrees.

FIGS. 29 and 30 show an embodiment of a fastener-less bar and clip construction. FIG. 29 is an upper view of the fastener-less embodiment and FIG. 30 shows the bar from a bottom perspective. The bar 2002 includes locating positions 2002a and 2002b on either end of the bar 2002. The locating positions 2002a and 2002b may be generally circular in shape. Around the periphery of the locating positions 2002a and 2002b may be depressions 2032. The depressions 2032 may be limited and set at predetermined angles, or they may be disposed all around the periphery of the locating positions 2002a and 2002b.

In this embodiment, the quick release clip 2016 is simply pushed downwardly into the locating positions 2002a and 2002b, and includes a protrusion 2030 and corresponds to the depression 2032. Corresponds in this context means that the protrusion is any shape and size that limits the relative rotational movement between the quick clip member 2016 and the bar 2002, while still allowing the quick clip member 2016 to fully seat within the locating positions 2002a and 2002b. For instance, the protrusion 2030 may be the same size and shape as the depression 2032. In another aspect of the present disclosure the protrusion may be the same shape but slightly larger than the depression 2032 to ensure a slight interference. In still other embodiments, the shape could be a cross, a star, a circle, or any other shape that allows the protrusion 2030 to contact the inner walls of the depression 2032 to prevent rotation. As a person of ordinary skill would readily appreciate, the protrusion 2030 and depression 2032 may be disposed on their opposite parts, e.g. the protrusion on the bar 2002 and the depression on the quick clip member 2016. The quick release clip 2016 could conceivably be biased using a spring through the center of the clip and holding surface 2012 into an engaged position and finger force applied upward to move the clip to a second or different position.

In the typical engagement, the quick clip member 2016 may further include clip wings 2020. The clip wings 2020 extend downwardly from the member 2016 and are slightly flexible toward the center point of the main portion of the member 2016. The quick clip member 2016 is pressed into one of the locating positions 2002a and 2002b by pushing the wings 2020 into the hole in the locating positions 2002a and 2002b. The wings 2020 have a beveled surface that forces the wings to flex inwardly as the member 2016 is pushed further into the hole within the locating positions. The beveled surfaces of the wings 2020 extend to an undercut 2010. As the undercut 2010 passes a holding surface 2012 of the locating positions, the wings 2020 extend outwardly toward their neutral position, although when attached to the bar the wings 2020 may not extend fully back out to the neutral position. The base portion of the wings 2020 may contact the inner wall of the hole of the locating positions 2002a and 2002b, and in this way keep the quick clip member 2016 centered within the hole and prevent lateral movement between the quick clip member 2016 and the bar 2002. The undercut 2010 may contact an under surface 2012 of the locating position 2016 and prevent the quick clip member 2016 from being pulled out from the bar 2002. To release the quick clip member 2016, a user simply squeezes the wings 2020 together such that the outer radius of the undercuts 2010 are inside of the hole of the locating positions and the member 2002 may be pulled out by hand and without the use of any separate tools.

In yet another aspect of the present disclosure is shown in FIG. 31 and FIG. 32. An adjustable bar 2102 is shown as two end bars 2012a and 2102b. The end bars 2102a and 2102b may be placed in an overlapping fashion and held in place by a bar lock 2120. The bar lock 2120 has a through slot 2126 that allows the end bars 2102a and 2102b to both fit within one on top of another. The bar lock 2120 may also include a threaded hole 2122 and a set screw 2124 therein. With the end bars 2102a and 2102b in place, the set screw 2124 may be tightened creating pressure on the bars 2102a and 2102b, forcing them against one wall of the through slot 2126, and holding them in place fixedly by the pressure applied by the set screw 2124. In this way, a practitioner may set one of any of a variety of lengths of the bar 2102 and customize for each user of the bar. The longest extent of the length of the adjustable bar 2102 would only be limited by the lengths of end bars 2102a and 2102b. The combined length may be adjusting the extent of the overlap of the bars 2102a, 2102b.

The quick clip members 2700 according to this aspect of the present disclosure may include a quick clip portion 2702, an intermediate portion 2710, and a bottom portion 2720. The three portions 2702, 2710, and 2720 may be assembled first and the angle of the quick clip portion 2702 set with respect to the bottom portion 2720 before introducing the bar 2102 into the assembly 2700. The assembly 2700 includes a slot 2730 into which the bar 2102 is placed, and a screw is tightened through the center holes of each part, through a hole 2104 of the bar 2102, and into a threaded portion of the bottom portion 2720. The intermediate portion may be a plastic material, but is most typically a thermoplastic elastomer (TPE), which is sometimes referred to as thermoplastic rubber, such that, when the intermediate portion 2710 is an elastomeric material, it provides higher friction and a better grip of the bar 2102 to prevent slippage during use if one wearing the device stands (see right side of assembly shown in FIG. 32). The intermediate portion 2710 typically has downwardly extending projection portions 2760, 2770 that extend through apertures the bottom portion and around the perimeter of the bottom portion to provide the gripping surface on the bottom thereof. The upwardly facing teeth 2740 on the inner portion of the bottom portion 2720 can function to grip the bottom surface of the intermediate portion 2710 and grip the elastomeric material. It should be known to one of ordinary skill that the bar may be placed into the bottom portion 2720 first and the intermediate portion 2710 and quick clip portion 2702 then placed on top of the bar 2102 before tightening screw 2750 as well.

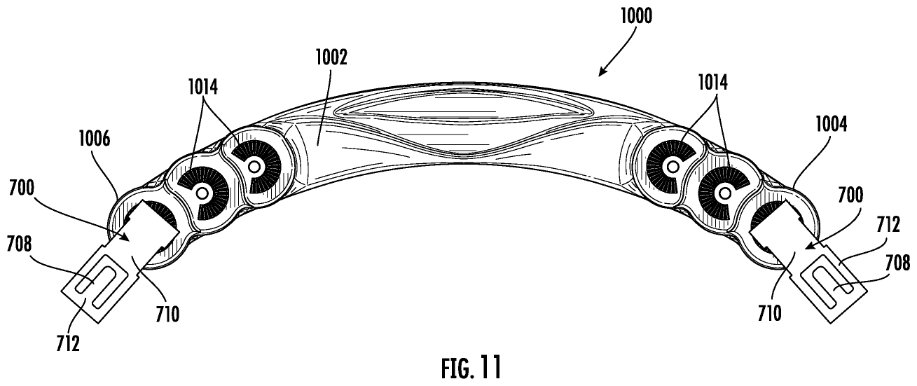

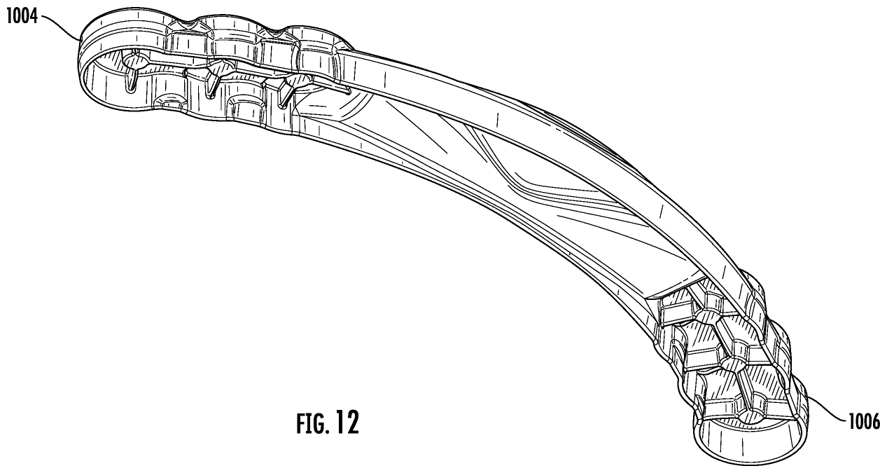

FIGS. 33-36 show an extendible version of a unitary bar 3000 assembly. In FIG. 35, there is a bar 3002 with the inner locating position 3008 and intermediate position 3010. This makes for a shorter bar assembly 3000 that does not extend past the shoes of the user very far. Once the user graduates to a longer bar assembly 3000, extensions 3012 may be attached to the bar 3002 through the connection holes 3018 much in the same way that the quick release members 700 are attached to the bar 1002 from the embodiment of FIG. 11, above. The extensions 3012 include the outer locating position 3012 and the corresponding quick release member connection hole 3018.

It will be understood by one having ordinary skill in the art that construction of the described disclosure and other components is not limited to any specific material. Other exemplary embodiments of the disclosure disclosed herein may be formed from a wide variety of materials, unless described otherwise herein.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the Applicant intends to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.”

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present disclosure, and further it is to be understood that such concepts are intended to be covered by the following claims unless these claims by their language expressly state otherwise.

What is claimed is:

1. An orthotic assembly for locating orthotic footwear to maintain a user's feet in a predetermined therapeutic position, comprising:
a unitary, curved orthotic bar having a first end and a second end opposite the first end;
a first inner locating position having a first inner connection hole on a top surface of the bar and proximate the first end;
a first outer locating position having a first outer connection hole on the top surface of the bar outside the first inner locating position and proximate the first end;
a second inner locating position having a second inner connection hole on the top surface of the bar and proximate the second end;
a second outer locating position having by a second outer connection hole on the top surface of the bar outside the second inner locating position and proximate the second end;
wherein each of the first inner locating position, the first outer locating position, the second inner location position, and the second outer locating position further comprise a plurality of ribs on top surface of the bar and at least partially surrounding and disposed in a radial arrangement around each of the first inner connection hole, the first outer connection hole, the second inner connection hole, and the second inner connection hole.

2. The orthotic assembly of claim 1 further comprising a first medial locating position having a first medial connection hole and a plurality of ribs spaced around the first medial connection hole and wherein the first medial locating position is between the first inner locating position and the first outer locating position and a second medial locating position having a second medial connection hold and a plurality of ribs spaced around the second medial connection hole and wherein the second medial locating position is between the second inner locating position and the second outer locating position.

3. The orthotic assembly of either of claim 1 or 2 further comprising a first quick release member engaged using a fastener to at least one of the first inner connection hole, the first medial connection hole and the first outer connection hole and a second quick release member engaged using a fastener to as least one of the second inner connection hole, the second medial connection hole and the second outer connection hole.

4. The orthotic assembly of claim 3, wherein both of the first quick release member and the second quick release member comprise a plurality of ribs configured to fit within and interfere with the plurality of ribs on the top surface of the bar.

5. The orthotic assembly of either claim 3 or 4 wherein the first quick release member and the second release member further comprise a threaded stud coupled on a bottom side of the quick release member.

6. The orthotic assembly of claim 5, wherein the plurality of ribs of the first quick release member on the bottom surface thereof and the plurality of ribs of the second quick release member are brought into interference with ribs on the top surface of the bar when a nut is attached and tightened to the threaded stud of each of the first quick release member and the second quick release member.

7. The orthotic assembly of any one of claims 3-6, wherein ribs on each of the quick release member and the ribs on the bar are configured to allow a user to tighten the quick release member in any position about 360 degrees.

8. The orthotic assembly of any of claims 3-7, wherein the quick release members further comprise a main body extending from an angular adjustment base having an inclined top surface.

9. The orthotic assembly of claim 8, wherein the main body is configured to be inserted into and held within an article of orthotic footwear.

10. The orthotic assembly of any of claims 1-9, wherein the connection hole further comprises a metal sleeve.

11. The orthotic assembly of claim 10, wherein the metal sleeve is insert molded as a unitary piece within the bar.

12. An assembly for locating footwear to maintain a user's feet in a predetermined therapeutic position, comprising:
a bar having a first end and a second end;
a first locating position on the first end;
a second locating position on the second end;
wherein each of the first locating position and the second locating position further comprises a plurality of ribs at least partially surrounding and disposed in a radial arrangement extending about a center point of a connection hole;
a first quick release member comprising a plurality of ribs disposed on a bottom side of the first quick release member that extend in a radial fashion about a stud extending downwardly from a base of the first quick release member;
a second quick release member comprising a plurality of ribs disposed on a bottom side of the second quick release member that extend in a radial fashion about a stud extending downwardly from a base of the second quick release member;
wherein the ribs on each of the first and second quick release member are configured to fit between and interfere with the ribs at each of the first and second locating positions, respectively.

13. The assembly of claim 12, further comprising a third locating position on the first end and disposed inboard from the first locating position.

14. The assembly of claim 13, further comprising a fourth locating position on the second end and disposed inboard from the second locating position.

15. The assembly of claim 14, wherein each of the third and fourth locating positions further comprises a plurality of ribs at least partially surrounding and disposed in a radial arrangement extending about a center point of a connection hole.

16. The assembly of claim 15, further comprising a fifth locating position on the first end and disposed inboard from the third locating position, and a sixth locating position on the second end and disposed inboard from the fourth locating position, wherein each of the fifth and sixth locating positions further comprises a plurality of ribs at least partially surrounding and disposed in a radial arrangement extending about a center point of a connection hole.

17. A sock comprising:
a main portion having an exterior facing away from a user's foot when the sock is worn by the user;
a plurality of anti-slip nubs disposed on an exterior of the main portion on a top, upward facing surface of the main portion of the sock;
wherein the plurality of anti-slip nubs are each configured to engage an interior surface of an ankle foot orthotic and interfere and prevent slippage of the user's foot with respect to an item of footwear attached to the assembly.

18. The sock of claim 17 further comprising an upwardly extending portion that extends upward from the ankle of the user's foot when being worn and over at least a portion of the bottom of the leg above the ankle wherein the upwardly extending portion has a front facing surface comprising a plurality of anti-slip nubs thereon.

19. A method of assembling a bar assembly for the treatment of clubfoot, comprising the steps of:
attaching a first quick release member to a first end of a bar by inserting a first stud disposed on the first quick release member through one of a first hole at a first locating position at the first end portion of the bar and a second hole in a second locating position at the first end of the bar;
attaching a second quick release member to a second end of the bar by inserting a second stud disposed on the second quick release member through one of a third hole at a third locating position proximate the second end of the bar and a fourth hole in a fourth locating position proximate the second end of the bar;
angularly locating the first quick release member by the steps of:
rotating the first quick release member until it reaches a desired angular location; and
tightening the first quick release member into engagement with the bar using a fastener and thereby bringing a first set of release member ribs into interference with a first set of bar ribs; and
angularly locating the second quick release member by the steps of:
rotating the second quick release member until it reaches a desired angular location; and
tightening the second quick release member into engagement with the bar and bringing a second set of release member ribs into interference with a second set of bar ribs.

20. The method of claim 19 further comprising the steps of:
attaching a pair of articles of footwear to a user;
assembling the pair of articles of footwear to the first and second quick release members by sliding main body portions disposed on each of the first and second quick release members into attachment receiving grooves in each of the pair of articles of footwear.

21. The method of either claim 19 or 20, wherein the tightening of each of the quick release members includes attaching a nut to the stud and tightening against a bottom side of the bar.

22. The method of any of claims 19-21, wherein the bar further includes a fifth locating position on the first end of the bar, and a sixth locating position on the second end of the bar.

23. The assembly of any of claims 1-18 in any combination.

24. A method of using the assembly of claims 1-18 to treat clubfoot in a human less than 5 years old using the Ponseti method.

25. A unitary, archuous, thermoplastic orthotic brace bar having multiple attachment locations for an orthotic brace at each of a first end and an opposing second end wherein each of the multiple attachment locations are configured to receive a quick release member that is configured to engage an orthotic brace by hand and without the use of tools and wherein each of the multiple attachment locations allow a user to engage the orthotic brace in an orientation to maintain a user's feet in a predetermined therapeutic position for the treatment of clubfoot.

26. An orthotic brace bar comprising a unitary archuous main body having a first end and a second end wherein each of the first end and the second end have an inner locating position having a threaded aperture and an outer locating position having a treaded aperture.

27. The orthotic brace bar of claim 26, wherein the inner locating position and the outer locating position each further comprises a plurality of ribs at least partially surrounding and disposed in a radial arrangement extending about a center point of a threaded connection hole.

28. The orthotic brace bar of any one of claim 26-28, wherein the inner locating position and the outer locating position are at different heights on the orthotic brace bar.

29. The orthotic brace bar of claim 28, wherein inner locating position is above the outer locating position on the first end and the second end of the orthotic brace bar.

30. The orthotic brace bar of claim 29, wherein the inner locating position and the outer locating position are each in substantially parallel planes with one another.

31. The orthotic brace bar of any of claims 25-30, wherein the orthotic brace bar is formed from a thermoplastic polymer.

32. The orthotic brace bar of claim 31, wherein the threaded connection hole is formed from a metal sleeve with threads on an interior facing surface thereof configured to engage a screw or bolt.

33. An orthotic brace for the treatment of clubfoot comprising:
a bar assembly consisting of a first bar portion having a distal end and a second bar portion having a distal end wherein the first bar portion and the second bar portion are substantially planar and longer than they are wide and wherein the first bar portion and the second bar portion overlap at least a portion of one another and an overlapping portion is defined by the portions of the first bar portion and the second bar portion that overlap;
a bar lock mechanism engaged to the overlapping portion of the bar assembly to fix the bar assembly at a user selected length;
a first bar portion aperture through the first bar portion proximate the distal end of the first bar portion;
a second bar portion aperture through the second bar portion proximate the distal end of the second bar portion;
a first quick release member and a second quick release member each having an angular base portion and a quick release tab extending away from the angular base portion and configured to engage a receiving channel on the bottom surface of an orthotic shoe;
a bottom mounting portion having a plurality of apertures therein;
an elastomeric intermediate portion having an engagement aperture through and a plurality of downwardly extending elastomeric extensions wherein the plurality of downwardly extending elastomeric extensions extend over an exterior surface of the bottom mounting portion, at least one of the plurality of apertures in the bottom mounting portion, or both; and
wherein the first quick release member, the bottom mounting portion and the elastomeric intermediate portion and one of the first bar portion aperture and the second bar portion aperture are engaged with one another using a fastener that passes through each of the first quick release member, the elastomeric intermediate member, at least one of the first bar portion aperture and the second bar portion aperture and into a threaded engagement cavity on a bar assembly facing side of the bottom mounting portion.

34. The orthotic brace of claim 33, wherein the threaded engagement cavity is an aperture that passes completely through the bottom mounting portion.

35. The orthotic brace of one of claim 33 or 34, wherein the non-elastomeric bottom mounting portion has a bar assembly facing surface that faces and engages a bottom surface of the bar and wherein the bar assembly facing surface has a plurality of teeth that extend above a top surface of first bar portion or the second bar portion when engaged therewith and wherein the non-elastomeric bottom portion a bar receiving channel therein shaped to matingly engage a bottom surface of the distal end portion of the first bar portion or the second bar portion proximate the distal ends thereof.

36. The orthotic brace of claim 35, wherein the bottom mounting portion is a non-elastomeric material.

37. The orthotic brace of any one of claim 33-34, wherein the bottom mounting portion is a non-elastomeric material.

38. The orthotic brace of any one of claims 33-34, wherein the bottom mounting portion is a plastic material.

39. The orthotic brace of claim 35, wherein the bottom mounting portion is a plastic material.

40. The orthotic brace of any one of claims 33-34, wherein the quick release tab of the first quick release member and the quick release tab of the second quick release member each extend away from the angular base portion in a plane that is parallel to the plane defined by the top surface of the angular base portion.

41. The orthotic brace of claim 35, wherein the quick release tab of the first quick release member and the quick release tab of the second quick release member each extend away from the angular base portion in a plane that is parallel to the plane defined by the top surface of the angular base portion.

42. The orthotic brace of claim 33, wherein the quick release tab of the first quick release member and the quick release tab of the second quick release member each extend away from the angular base portion in a plane that is parallel to the plane defined by the top surface of the angular base portion and wherein the bottom mounting portion is a non-elastomeric material.

43. The orthotic brace of claim 33, wherein the quick release tab of the first quick release member and the quick release tab of the second quick release member each extend away from the angular base portion in a plane that is parallel to the plane defined by the top surface of the angular base portion and wherein the bottom mounting portion is a plastic molded material.

44. The orthotic brace of any one of claims 33-43, wherein the quick release tab of the first quick release member and the quick release tab of the second quick release member each extend away from the angular base portion in a plane that is the same as a plane defined by the top surface of the angular base portion.

45. The orthotic brace of any one of claims 41-44, wherein a top surface of the angular base portion is at an angle that is between from 10 to 20 degrees from the horizontal plane of the bottom surface of the angular base portion of the quick release member.

46. The orthotic brace of any one of claims 41-45, wherein the top surface of the angular base portion is at an angle that is between 10 and 15 degrees from the horizontal plane of the bottom surface of the angular base portion of the quick release member.

47. The orthotic brace of any one of claims 41-46, wherein the top surface of the angular base portion is at an angle that is either 10 or 15 degrees from the horizontal plane of the bottom surface of the angular base portion of the quick release member.
